(19) United States
(12) Patent Application Publication
(10) Pub. No.: US 2020/0279614 A1
(43) Pub. Date: Sep. 3, 2020

(54) CLUMP PATTERN IDENTIFICATION IN CANCER PATIENT TREATMENT

(71) Applicant: International Business Machines Corporation, Armonk, NY (US)

(72) Inventors: Filippo Utro, Pleasantville, NY (US); Chaya Levovitz, New York, NY (US); Laxmi Parida, Mohegan Lake, NY (US); Kahn Rhrissorrakrai, Woodside, NY (US)

(21) Appl. No.: 16/288,371

(22) Filed: Feb. 28, 2019

(51) Int. Cl. G16B 20/40; G16B 40/00; G16B 10/00; G16B 50/00; G16B 20/20; G16B 30/00

(52) U.S. Cl. CPC G16B 20/40; G16B 40/00; G16B 30/00; G16B 50/00; G16B 20/20; G16B 10/00

(57) ABSTRACT

A computer-implemented method includes inputting, to a processor, genomic data from a plurality of subjects, the genomic data including first sample genomic data prior to a treatment, and second sample genomic data after the treatment; determining, by the processor, a plurality of δ's for the plurality of subjects, wherein each δ is a genetic change in the second sample compared to the first sample genomic data; creating, by the processor, a matrix of the plurality of subjects and their features which features are the genetic changes or clusters of genetic changes in the plurality of δ's of the subjects; biclustering, by the processor, the matrix of the plurality of subjects and their features, to provide clumps of subjects sharing a common feature such as a shared genetic change or shared cluster of genetic changes; and outputting, by the processor, the clumps of subjects, the common features, and the treatment.

### BACKGROUND

The present invention generally relates to computing systems, and more specifically, to computer systems, computer-implemented methods, and computer program products configured to electronically implement determination of patterns of genetic alterations in response to treatment such as cancer treatment.

Genetic alterations in subjects undergoing cancer treatment can impact the favorable/unfavorable response to a treatment. Identifying genetic alterations impacting drug response remains challenging since only several of them have been validated and often are hidden in large complex sequencing datasets. Despite these challenges, the identification of such genetic alterations can be used to predict outcomes and guide therapy.

### SUMMARY

Embodiments of the present invention are directed to a computer-implemented method comprising inputting, to a processor, genomic data from a plurality of subjects, wherein the genomic data for each subject of the plurality of subjects comprises first sample genomic data from a first sample taken prior to a treatment, and second sample genomic data from a second sample taken after the treatment; determining, by the processor, a plurality of δ's for each of the plurality of subjects, wherein each δ is a genetic change in the second sample genomic data compared to the first sample genomic data; creating, by the processor, a matrix of the plurality of subjects and their features, wherein the features comprise the genetic changes or clusters of genetic changes in the plurality of δ's for each of the plurality of subjects; biclustering, by the processor, the matrix of the plurality of subjects and their features, to provide clumps of subjects, each clump of subjects sharing a common feature, wherein the common feature is a shared genetic change or shared cluster of genetic changes; and outputting, by the processor, the clumps of subjects, the common features, and the treatment.

Embodiments of the invention are directed to computer program products and computer systems having substantially the same features of the computer-implemented method described above.

Additional technical features and benefits are realized through the techniques of the present invention. Embodiments and aspects of the invention are described in detail herein and are considered a part of the claimed subject matter. For a better understanding, refer to the detailed description and to the drawings.

### BRIEF DESCRIPTION OF THE DRAWINGS

The specifics of the exclusive rights described herein are particularly pointed out and distinctly claimed in the claims at the conclusion of the specification. The foregoing and other features and advantages of the embodiments of the invention are apparent from the following detailed description taken in conjunction with the accompanying drawings in which:

FIG. 1 is an illustration of the time course of cancer treatment and biopsy sampling for a patient;

FIG. 2 illustrates a computer system for determining a common feature for the treatment according to embodiments of the present invention;

FIG. 3 is a flowchart of a computer-implemented method for determining a common feature for a treatment according to embodiments of the present invention;

FIG. 4 depicts a computer/processing system having components and/or functionality for practicing one or more embodiments of the present invention;

FIG. 5 depicts an illustration of the clumps of subjects with chronic lymphocytic leukemia (CLL) according to embodiments of the present invention;

FIG. 6 depicts a confirmation that the identified clumps of FIG. 5 are mathematically real according to embodiments of the present invention;

FIG. 7 depicts an illustration of the clumps of subjects with CLL according to embodiments of the present invention;

FIG. 8 depicts a confirmation that the identified clumps of FIG. 7 are mathematically real according to embodiments of the present invention;

FIG. 9 depicts the common features for a clump of three subjects according to embodiments of the present invention.

The diagrams depicted herein are illustrative. There can be many variations to the diagram or the operations described therein without departing from the spirit of the invention. For instance, the actions can be performed in a differing order or actions can be added, deleted or modified. Also, the term “coupled” and variations thereof describes having a communications path between two elements and does not imply a direct connection between the elements with no intervening elements/connections between them. All of these variations are considered a part of the specification.

In the accompanying figures and following detailed description of the described embodiments, the various elements illustrated in the figures are provided with two or three digit reference numbers. With minor exceptions, the leftmost digit(s) of each reference number correspond to the figure in which its element is first illustrated.

### DETAILED DESCRIPTION

Various embodiments of the invention are described herein with reference to the related drawings. Alternative embodiments of the invention can be devised without departing from the scope of this invention. Various connections and positional relationships (e.g., over, below, adjacent, etc.) are set forth between elements in the following description and in the drawings. These connections and/or positional relationships, unless specified otherwise, can be direct or indirect, and the present invention is not intended to be limiting in this respect. Accordingly, a coupling of entities can refer to either a direct or an indirect coupling, and a positional relationship between entities can be a direct or indirect positional relationship. Moreover, the various tasks and process steps described herein can be incorporated into a more comprehensive procedure or process having additional steps or functionality not described in detail herein.

The following definitions and abbreviations are to be used for the interpretation of the claims and the specification. As used herein, the terms “comprises,” “comprising,” “includes,” “including,” “has,” “having,” “contains” or “containing,” or any other variation thereof, are intended to cover a non-exclusive inclusion. For example, a composition, a mixture, process, method, article, or apparatus that comprises a list of elements is not necessarily limited to only those elements but can include other elements not expressly listed or inherent to such composition, mixture, process, method, article, or apparatus.

Additionally, the term “exemplary” is used herein to mean “serving as an example, instance or illustration.” Any embodiment or design described herein as “exemplary” is not necessarily to be construed as preferred or advantageous over other embodiments or designs. The terms “at least one” and “one or more” can be understood to include any integer number greater than or equal to one, i.e. one, two, three, four, etc. The terms “a plurality” can be understood to include any integer number greater than or equal to two, i.e. two, three, four, five, etc. The term “connection” can include both an indirect “connection” and a direct “connection.”

The terms “about,” “substantially,” “approximately,” and variations thereof, are intended to include the degree of error associated with measurement of the particular quantity based upon the equipment available at the time of filing the application. For example—“about” can include a range of ±8% or 5%, or 2% of a given value.

For the sake of brevity, conventional techniques related to making and using aspects of the invention may or may not be described in detail herein. In particular, various aspects of computing systems and specific computer programs to implement the various technical features described herein are well known. Accordingly, in the interest of brevity, many conventional implementation details are only mentioned briefly herein or are omitted entirely without providing the well-known system and/or process details.

Turning now to an overview of technologies that are more specifically relevant to aspects of the invention, it is known that genetic alterations in a subject undergoing treatment for cancer, for example, can impact the response to treatment, e.g., drug therapy. Responses can be either favorable or unfavorable. In general, any response to treatment is expected to have a genomic connection. Identifying the genetic alterations that impact treatment response remains challenging, as the gene alterations of interest are often hidden in large complex sequence databases. What is needed are novel methods for identifying genetic alterations associated with response to drug therapy.

Several definitions are provided.

As used herein, genomic data comprises DNA sequence information and/or gene identification for at least a portion of the genome of a subject, or the microbiome of a subject. High throughput or next generation sequencing allows for the sequencing of entire genomes using a massively parallel process in which multiple genome fragments are sequenced at once. Sequencing includes, for example mRNA sequencing. Genomic data may include identified genes and their variants, as well as expression levels of genes and variants such as log2 expression ratios. Genomic data optionally includes the cancer cell fraction (CCF) which is the fraction of cancer cells with a particular variant, the variant allele frequency (VAF) which is the relative frequency of a variant in a population expressed as a fraction or percentage, and/or the copy number variation (CNV) which is when the number of copies of a particular gene varies.

As used herein, a cluster of genes is a group of genes that are functionally related. For example, the members of cluster of genes may be in the same biological pathway.

As used herein, biclustering is a method of simultaneously clustering rows and columns of a matrix.

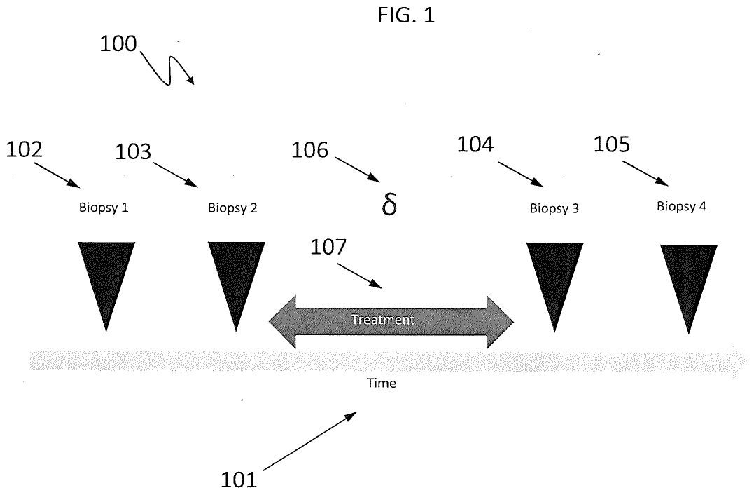

FIG. 1 illustrates the time course of cancer treatment and biopsy sampling for a patient according to an aspect of the invention. As illustrated in FIG. 1, during the time axis 101 of cancer identification and treatment, biopsy samples can be taken before 102, 103 and after 104, 105 a cancer treatment 107. The term δ 106 represents a genetic change in the patient's genomic data between a post-treatment time point 104, 105 and a pre-treatment time point 102,103. The δ can be, for example, a change in a gene or a cluster of genes in the patient's genome or microbiome.

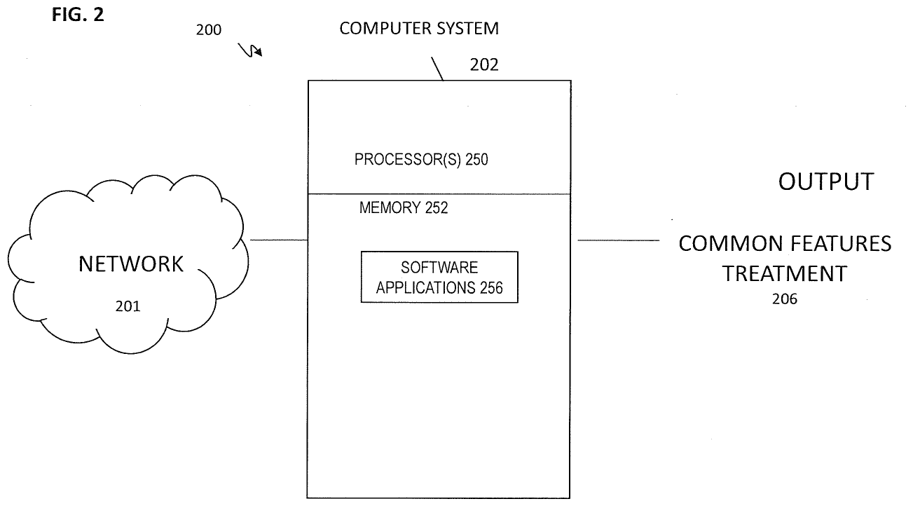

Turning now to a more detailed description of aspects of the present invention, an implementation of methods performed by, e.g., a computer system 202 depicted in FIG. 2 according to embodiments of the invention will now be described.

More specifically, aspects of the computer-implemented method executed by the system 200 and software application 256 are illustrated in FIG. 2. Genomic data from a plurality of subjects are inputted to processor 250. The common feature for the treatment is determined e.g., by processor 250 and software applications 256 depicted in FIG. 2. The common feature for the treatment can be used to determine outcomes 206 for the subject.

The processor 250 executes the software application 256 (depicted in FIG. 2) which includes the model and optional assumptions. The input to the processor 250 in the method is genomic data from a plurality of subjects, wherein the genomic data for each subject of the plurality of subjects comprises first sample genomic data from a first sample taken prior to a cancer treatment, and second sample genomic data from a second sample taken after the treatment

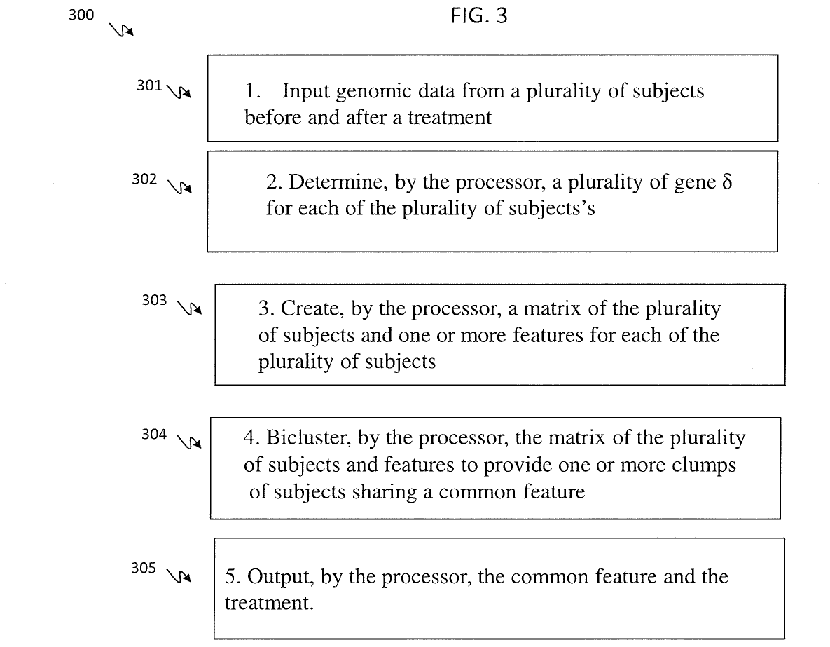

A flow chart of the method 300 is detailed below and shown in FIG. 3.

The input to the method is genomic data from a plurality of subjects 301. The genomic data, which is inputted to the processor 250, comprises first sample genomic data from a first sample taken prior to a treatment, and second sample genomic data from a second sample taken after the treatment. The sample taken after treatment can be taken at an early stage or a late stage after treatment, for example. Without being held to theory, it is believed that virtually any administered treatment can have a detectable effect on the genome of a subject. For example, any treatment can have a “selective pressure” on cells and can cause genomic changes.

In an aspect, the genomic data is from the genomes of the subjects. In this aspect, the treatment can be a cancer treatment and the sample can be a biopsy sample comprising cancer cells from the subjects.

In another aspect, the genomic data is from the microbiomes of the subjects. Any type of administered drug can have an effect on the microbiome of a subject. A subject's microbiome can shift as a result of many specific and non-specific biological changes experienced by the patient, and thus this dysbiosis (microbiome change) can be a result of any treatment such as cancer treatment including chemotherapy, radiation therapy, targeted drug therapy, and immunotherapy. Changes in the microbiome status can indicate drug resistance, drug response, and drug intolerance (adverse effects). In this aspect, the treatment can be an antibiotic treatment, cancer treatment, immunotherapy, and the sample can be from the gastrointestinal tract of the subject. Additional drug types that have been shown to affect the microbiome include acetaminophen, statins, cardiac drugs, antidiabetic drugs and others. Any type of drug can have an effect on the microbiome.

The method then includes determining 302, by the processor 250, a plurality of δ's for each of the plurality of subjects, wherein each δ is a genetic change in the second sample genomic data compared to the first sample genomic data. Essentially, each δ represents a change in the subjects' genomic data resulting from the treatment. Each patient can essentially be represented as a signature or vector of δ's.

In aspects of the invention, the genetic change comprises a presence of at least one gene; an absence of the least one gene; a sequence variation of at least one gene; or an expression level change of at least one gene.

The δ 106 is optionally determined with certain noise thresholds based on an overall distribution of δ values, such as assigning p-values or assigning a lower value to the S. The δ's can be used either directly or with pre-processing.

In an aspect, the δ's can be pre-processed to a binary representation of the gene expression matrix. When the δ's are genes, the binarized values can be used for from the matrix and bicluster, per steps 303 and 304. The binarized value can be used as the value for subsequent steps (matrix formation and clustering).

When considering gene pathways, for example, gene clusters that constitute the pathway can be evaluated based on their δ's. The proportion of the pathway with genes whose δ value passes a given threshold, e.g., a specified noise threshold based on an overall distribution of δ values, is then used for biclustering. This proportion may be, but is not limited to, enrichment values normalized for gene set size, percentage significant δ's, and the like. These enrichment values may then be used for biclustering, either directly or binarized based on overall distribution of enrichment values.

Next in 303, a matrix of the plurality of subjects and one or more features for each of the plurality of subjects is created by the processor 250, wherein the features comprise the genetic changes or clusters of genetic changes in the plurality of δ's for each of the plurality of subjects. In general, when forming a matrix of genetic changes for a cancer treatment for example, all of the genetic changes are in cancer genes. However, when forming a matrix of clusters of genetic changes, the clusters can include all genetic changes in a particular pathway whether or not they are changes in cancer genes.

Then in 304, the matrix of the plurality of subjects and their features is biclustered by the processor 250, to provide clumps of subjects, each clump of subjects sharing a common feature, wherein the common feature is a shared genetic change or shared cluster of genetic changes. Biclustering allows the identification of clumps of subjects with the same genes or gene clusters affected by the treatment.

The clumps of subjects sharing a common feature can be verified as mathematically real by performing permutation testing by methods known in the art. In an aspect, the method further comprises, permuting, by the processor, the matrix of subjects of their features and re-biclustering, by the processor, the permuted matrix to provide permuted clumps of subjects. An example of a permutation is to permute the actual data by shuffling, and then recomputing the bicluster results. This can be performed repeatedly, such as 10000 times. For each permuted bicluster results, the number of clusters, mean number of patients in clusters, mean number of features in clusters and mean area of clusters ((number of patients)*(number of features)) can be computed, by the processor. Each of these observations provides a distribution over the 10000 permutations from which empirical p-values can be calculated, by the processor, when results from actual data are compared against this distribution of permuted controls, by the processor. This is one form of permuted control to develop and empirical measure of significance.

Finally, in 305 the clumps of subjects, their common features, and the treatment are outputted by the processor 250. The common feature can be used to identify a common mechanism of response to treatment. For example, if it is found that gene ESR1 is a common feature for only patients that show resistance when given a particular treatment, such as Tamoxifen, is given, but is absent for patients that are responsive to Tamoxifen, the data suggests that ESR1 may be a mechanism of resistance to Tamoxifen and should be targeted to overcome resistance and restore sensitivity. In this example, ESR1 as a common feature suggests that there is a selective pressure for this gene to acquire or lose alterations, in response to treatment. Test subjects with an unknown mechanism of response to treatment can be mapped to patients with a known mechanism of response to treatment.

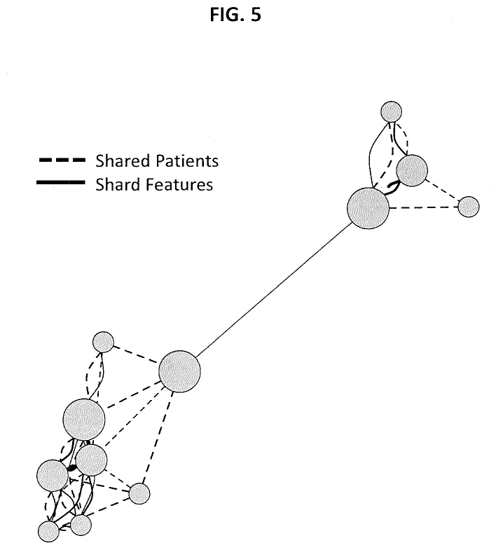

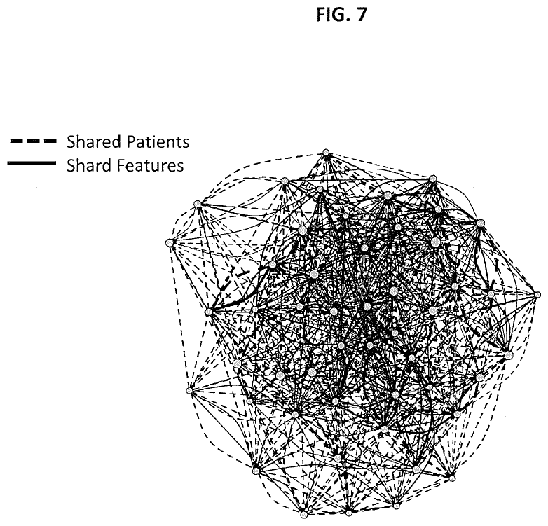

In another aspect, the method can further comprise connecting, by the processor, the clumps of subjects by a feature edge for clumps that share features, a subject edge for clumps that share subjects, or a combination thereof. Such representations are shown in FIG. 5 and FIG. 7. This representation enables the identification of common mechanisms (shared features) between sub-populations of the cohort (clumps of subjects) and may represent the core molecular machinery to yield the phenotypes that can be associated to each clump. Furthermore, additional features in clumps that are not in the shared edge may represent genetic modifiers to the core machinery to yield the potential phenotypic differences between connected clumps. Shared patient edges indicate patients with potentially complex or heterogenous phenotypes as they have aspects of different clumps, perhaps suggesting multiple mechanisms underlying the phenotype of interesting. For instance, they may have multiple response or resistance mechanisms to the tested treatment, or may have multiple lesions of the cancer of interest, each with different molecular profiles.

In an aspect of the invention, the method can further comprise comparing, by the processor, genomic data for a new patient subjected to the treatment with the common feature for the clump of subjects, and if the new patient genomic data shares the common feature, determining that the new subject and the clump of subjects have a same mechanism of response to the treatment. The method optionally further comprises determining, by the computer, a further treatment for the new patient based upon the same mechanism of response to the treatment. The further treatment can then be administered to the subject.

In an aspect, the common feature can be used to correlate the common feature and a phenotype for the clump of subjects. For example, the common feature may correspond to a pathway which can be associated with an observed phenotype, such as response to treatment, cellular phenotypes such as expressed cell surface receptors and other markers, blood markers, lymphocyte counts, tumor progression, and the like. For example, resistance to therapy that is after treatment is given the tumor, can “evolve” by creating “new” genomic changes to make the tumor less susceptible to the administered treatment. It is these “new” mutations that are identified by the methods described herein. When considering the δ's in the context of pathways/genesets, the functional resistance occurring can be better understood and targeted with an additional therapy to rescue the response.

In an aspect of the invention, the treatment is a cancer treatment. Exemplary cancer treatments include administration of a chemotherapeutic agent, radiation therapy, surgery, chemotherapy, targeted therapy, hormone therapy, immunotherapy, stem cell transplant, or a combination including at least one of the foregoing.

In an aspect of the invention, the method can further comprise comparing, by the processor, genomic data for a new patient subjected to the cancer treatment with the common feature for the plurality of subjects, and if the new patient genomic data shares the common feature, determining that the new subject and the plurality of subjects have a same mechanism of response to the cancer treatment. The method optionally further comprises determining, by the computer, a further treatment for the new patient based upon the mechanism of response to the cancer treatment. The further cancer treatment can then be administered to the subject.

Exemplary further cancer treatments include administering a signal transduction pathway inhibitor, an antimetabolite, an antimicrotubule agent, an alkylating agent, a nitrogen mustard, a nitrosourea, a platinum agent, an anthracycline, an antibiotic, a topoisomerase inhibitor, an alkyl sulfonate, a triazine, an ethyenimine, a folic acid analog, a pyrimidine analogue, a purine analog, an antitumor antibiotic, a hormone, an anti-angiogenic agent, an immunotherapeutic agent, a cell cycle signaling inhibitor, or a combination including one or more of the foregoing.

More specifically, further treatment thus include signal transduction pathway inhibitors (e.g., ErbB inhibitors, EGFR inhibitors such as erlotinib), antimetabolites (e.g., 5-fluoro-uracil, methotrexate, fludarabine), antimicrotubule agents (e.g., vincristine, vinblastine, taxanes such as paclitaxel, docetaxel), an alkylating agent (e.g., cyclophosphamide, melphalan, biochoroethylnitrosurea, hydroxyurea), nitrogen mustards, (e.g., mechloethamine, melphan, chlorambucil, cyclophosphamide and Ifosfamide); nitrosoureas (e.g., carmustine, lomustine, semustine and streptozocin;), platinum agents (e.g., cisplatin, carboplatin, oxaliplatin, JM-216, C 1-973), anthracyclines (e.g., doxrubicin, daunorubicin), antibiotics (e.g., mitomycin, idarubicin, adriamycin, daunomycin), topoisomerase inhibitors (e.g., etoposide, camptothecins), alkyl sulfonates including busulfan; triazines (e.g., dacarbazine); ethyenimines (e.g., thiotepa and hexamethylmelamine); folic acid analogs (e.g., methotrexate); pyrimidine analogues (e.g., 5 fluorouracil, cytosine arabinoside); purine analogs (e.g., 6-mercaptopurine, 6-thioguanine); antitumor antibiotics (e.g., actinomycin D; bleomycin, mitomycin C and methramycin); hormones and hormone antagonists (e.g., tamoxifen, cortiosteroids), anti-angiogenic agents (bevacizumab, endostatin and angiostatin), immunotherapeutic agents (transfection with cytokines such as interleukin 2, interleukin 4 or granulocyte-macrophage colony stimulating factor), cell cycle signaling inhibitors (CDK2, CDK4, and CDK6 inhibitors) and any other cytotoxic agents, (e.g., estramustine phosphate, prednimustine).

For example, signal transduction inhibitors include inhibitors of receptor tyrosine kinases, non-receptor tyrosine kinases, SH2/SH3domain blockers, serine/threonine kinases, phosphotidyl inositol-3 kinases, myo-inositol signaling, and Ras oncogenes. Growth factor receptor tyrosine kinases include, for example, epidermal growth factor receptor (EGFr), platelet derived growth factor receptor (PDGFr), erbB2, erbB4, ret, vascular endothelial growth factor receptor (VEGFr), tyrosine kinase with immunoglobulin-like and epidermal growth factor homology domains (TIE-2), insulin growth factor-I (IGFI) receptor, macrophage colony stimulating factor (cfms), BTK, ckit, cmet, fibroblast growth factor (FGF) receptors, Trk receptors (TrkA, TrkB, and TrkC), ephrin (eph) receptors, and the RET protooncogene. Tyrosine kinases, which are not growth factor receptor kinases are termed non-receptor tyrosine kinases. Non-receptor tyrosine kinases include cSrc, Lck, Fyn, Yes, Jak, cAbl, FAK (Focal adhesion kinase), Brutons tyrosine kinase, and Bcr-Abl.

Inhibitors of Serine/Threonine Kinases include MAP kinase cascade blockers which include blockers of Raf kinases (rafk), Mitogen or Extracellular Regulated Kinase (MEKs), and Extracellular Regulated Kinases (ERKs); and the Protein kinase C family member blockers including blockers of PKCs (alpha, beta, gamma, epsilon, mu, lambda, iota, zeta). IkB kinase family (IKKa, IKKb), PKB family kinases, akt kinase family members, and TGF beta receptor kinases.

Inhibitors of Phosphotidyl inositol-3 Kinase family members including blockers of PI3-kinase, ATM, DNA-PK, and Ku.

Inhibitors of Ras Oncogene include inhibitors of farnesyltransferase, geranyl-geranyl transferase, and CAAX proteases as well as anti-sense oligonucleotides, ribozymes and immunotherapy.

Alkylating agents alkylate molecules such as proteins, RNA and DNA and can covalently bind these molecules.

Alkylating agents affect any point in the cell cycle and thus are known as cell cycle-independent drugs.

Antimetabolites impede DNA and RNA synthesis.

Anti-microtubule agents block cell division by preventing microtubule function.

In an aspect, a computer program product for generating a clump of subjects with a common feature resulting from a treatment comprises a computer readable storage medium having program instructions embodied therewith, the program instructions executable by a processor to cause the processor to perform operations as described above in the computer-implemented method.

In another aspect, a system for generating a clump of subjects with a common feature resulting from a treatment comprises: a processor; and a computer readable storage medium storing comprising executable instructions that, when executed by the processor, cause the processor to perform operations as described above in the computer-implemented method.

FIG. 2 depicts a system 200 according to embodiments of the invention. Network 201 and computer system 202 can be used to store and communicate genomic data from a plurality of subjects, to determine a δ for each of the plurality of subjects, to create a matrix of the plurality of subjects and one or more features for each of the plurality of subjects, the bicluster the matrix, and to output the common feature and the cancer treatment. The common feature and the cancer treatment are used to make a treatment decision 206 which can then be administered to a patient. The computer system 202 includes one or more processors 250, memory 252, and one or more software applications 256 having computer-executable instructions to function as discussed herein. The processors 250 are configured to the execute computer-executable instructions of the software applications 256.

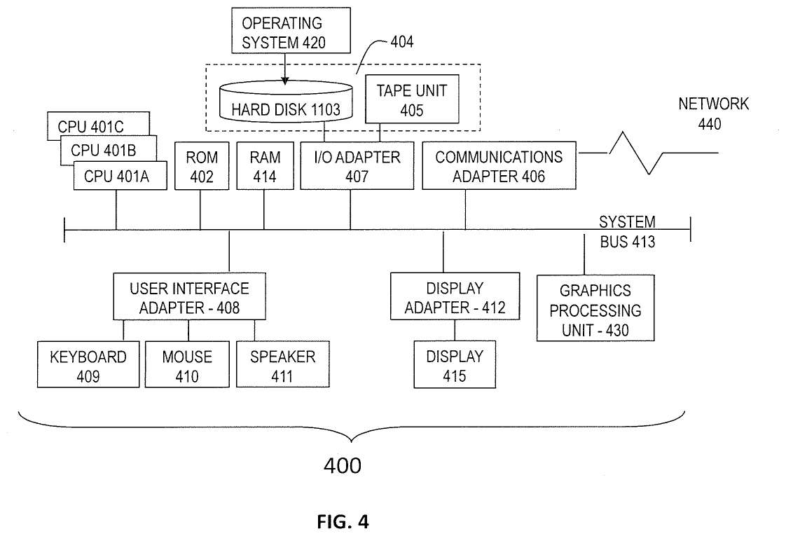

FIG. 4 depicts exemplary components of a computer system 400 according to one or more embodiments of the present invention. Any of the elements and functionality of computer system 400 can be included in any of the elements in FIGS. 1-3 and 5-9. Particularly, computer system 202 can implement the elements of computer system 1100 to perform the functions discussed herein. The computer system 200 is a processing system. The processing system 400 can include one or more central processing units (processors) 401A, 401B, 401C, etc. (collectively or generically referred to as processor(s) 401). In one or more embodiments, each processor 401 can include a reduced instruction set computer (RISC) microprocessor. Processors 401 are coupled to system memory 414 and various other components via a system bus 413. Read only memory (ROM) 402 is coupled to the system bus 413 and can include a basic input/output system (BIOS), which controls certain basic functions of processing system 400.

FIG. 4 further depicts an input/output (I/O) adapter 407 and a network adapter 406 coupled to the system bus 413. I/O adapter 407 can be a small computer system interface (SCSI) adapter that communicates with a hard disk 403 and/or tape storage drive 405 or any other similar component. I/O adapter 407, hard disk 403, and tape storage device 405 are collectively referred to herein as mass storage 404. Operating system 420 for execution on the processing system 400 can be stored in mass storage 404. The network adapter 406 interconnects bus 413 with an outside network, for example, network 440, enabling data processing system 400 to communicate with other such systems. A screen (e.g., a display monitor) 415 is connected to system bus 413 by display adaptor 412, which can include a graphics adapter to improve the performance of graphics intensive applications and a video controller. In one or more embodiments of the present invention, adapters 407, 406, and 412 can be connected to one or more I/O busses that are connected to system bus 413 via an intermediate bus bridge (not shown). Suitable I/O buses for connecting peripheral devices such as hard disk controllers, network adapters, and graphics adapters typically include common protocols, such as the Peripheral Component Interconnect (PCI). Additional input/output devices are shown as connected to system bus 413 via user interface adapter 408 and display adapter 412. A keyboard 409, mouse 410, and speaker 411 all interconnected to bus 413 via user interface adapter 408, which can include, for example, a Super I/O chip integrating multiple device adapters into a single integrated circuit.

In exemplary embodiments, the processing system 400 includes a graphics processing unit 430. Graphics processing unit 430 is a specialized electronic circuit designed to manipulate and alter memory to accelerate the creation of images in a frame buffer intended for output to a display. In general, graphics processing unit 430 is very efficient at manipulating computer graphics and image processing and has a highly parallel structure that makes it more effective than general-purpose CPUs for algorithms where processing of large blocks of data is done in parallel.

Thus, as configured in FIG. 4, the processing system 400 includes processing capability in the form of processors 401, storage capability including system memory 414 and mass storage 404, input means such as keyboard 409 and mouse 410, and output capability including speaker 411 and display 415. In one implementation, a portion of system memory 414 and mass storage 404 collectively store an operating system coordinate the functions of the various components shown in FIG. 4.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instruction by utilizing state information of the computer readable program instructions to personalize the electronic circuitry, in order to perform aspects of the present invention.

The invention is further illustrated by the following non-limiting examples:

### EXAMPLES

### Example 1

### Gene Level Analysis of Patients with Chronic Lymphocytic Leukemia (CLL)

The method as shown in FIG. 3 was applied to a population of patients with CLL administered a BCL-2 inhibitor as the cancer treatment. Genomic data was obtained from public databases. FIG. 5 shows an illustration of the clumps of subjects as identified using the methods described herein. The nodes (gray circles) are the biclusters, that is, the clumps of patients with common features. They can be connected by two types of edges. 1) Solid line—drawn if two biclusters share features, e.g. genes or genesets. 2) Dashed line—drawn if two biclusters share patients. Edge thickness is proportional to the amount shared between the node. This provides an accurate representation of the relatedness between biclusters. Or said differently, the biclusters represent patients with common features and then the graph represents how those different groups of patients relate to one another.

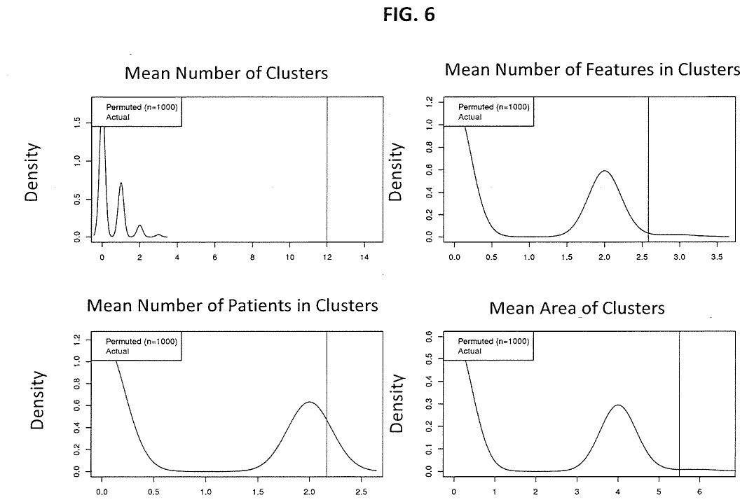

FIG. 6 shows a confirmation that the identified clumps are mathematically real. The plots are the permutation tests and empirical p-value calculations. The data was permuted by shuffling, and then recomputing the bicluster results. This was performed 10000 times. For each permuted bicluster results, the number of clusters, mean number of patients in clusters, mean number of features in clusters and mean area of clusters ((number of patients)*(number of features)) were computed, by the processor. Each of these observations provided a distribution over the 10000 permutations from which empirical p-values were calculated, by the processor, when results from actual data were compared against this distribution of permuted controls, by the processor. Each panel thus represents a particular bicluster metric: number of clusters, mean number of patients in clusters, mean number of features in clusters and mean area of clusters ((number of patients)*(number of features)). And the straight line is the actual data. So we can see that for each of these metrics the straight line falls to the right of the means of these distributions. The δp-value≤0.05 and CNV≥4 in case of CNV.

### Example 2

### Gene Cluster/Pathway Level Analysis of Patients with Chronic Lymphocytic Leukemia (CLL)

The analysis of the same subjects as Example 1 was repeated using a gene cluster analysis. FIG. 7 shows an illustration of the clumps of subjects as identified using the methods described herein. Specific patients with similar responses group together and suggest a potential mechanism of response. The nodes (gray circles) are the biclusters, that is, the clumps of patients with common features. They can be connected by two types of edges. 1) Solid line—drawn if two biclusters share features, e.g. genes or genesets. 2) Dashed line—drawn if two biclusters share patients. Edge thickness is proportional to the amount shared between the node. This provides an accurate representation of the relatedness between biclusters. Or said differently, the biclusters represent patients with common features and then the graph represents how those different groups of patients relate to one another.

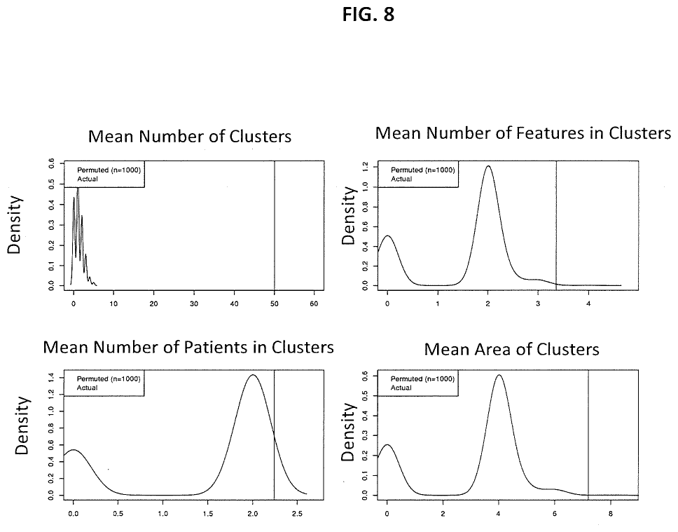

FIG. 8 shows a confirmation that the identified clumps are mathematically real. The plots are the permutation tests and empirical p-value calculations. The data was permuted by shuffling, and then recomputing the bicluster results. This was performed 10000 times. For each permuted bicluster results, the number of clusters, mean number of patients in clusters, mean number of features in clusters and mean area of clusters ((number of patients)*(number of features)) were computed, by the processor. Each of these observations provided a distribution over the 10000 permutations from which empirical p-values were calculated, by the processor, when results from actual data were compared against this distribution of permuted controls, by the processor. Each panel thus represents a particular bicluster metric: number of clusters, mean number of patients in clusters, mean number of features in clusters and mean area of clusters ((number of patients)*(number of features)). And the straight line is the actual data. So we can see that for each of these metrics the straight line falls to the right of the means of these distributions. The δ p-value≤0.05 and CNV≥4 in case of CNV.

### Example 3

### Clump Analysis of Unknown Patent

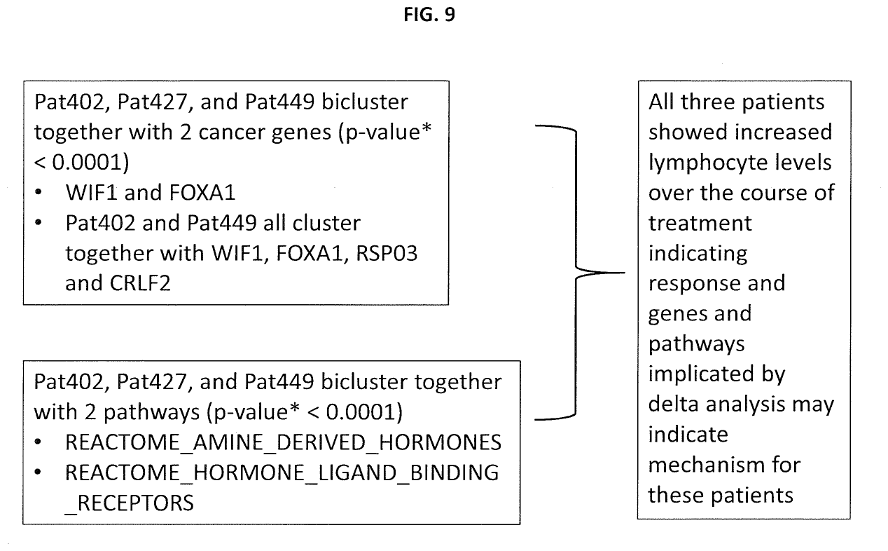

Genomic data for unknown patient Pt402 was compared to the common feature for the plurality of subjects Pt402, Pt427 and Pt449 from the analysis of Examples 1 and 2. Subjects Pt402, Pt427 and Pt449 blicluster together with regard to two cancer genes, WIF1 and FOXA1, and two gene clusters, REACTOME_AMINE_DERIVED_HORMONES and REACTOME_HORMONE_LIGAND_BINDING_RECEPTORS as shown in FIG. 9. The commonality in bicluster analysis reveals a possible outcome interpretation for the unknown patient. For example, it was reported that these patients show increased lymphocyte levels over the course for treatment indicating these tumors are immunologically ‘hot’ and possibly primed for immunotherapies. The clinically observed increase in lymphocytes is consistent with the genes and gene clusters identified by the analysis and suggest these genes and gene clusters may be the potential molecular mechanism for the lymphocytic increase. Thus treatments that target these genes and/or gene clusters could be given priority consideration for these patients.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present invention have been presented for purposes of illustration, but are not intended to be exhaustive or limited to the embodiments described. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments described herein.

What is claimed is:

1. A computer-implemented method comprising:
inputting, to a processor, genomic data from a plurality of subjects, wherein the genomic data for each subject of the plurality of subjects comprises first sample genomic data from a first sample taken prior to a treatment, and second sample genomic data from a second sample taken after the treatment;
determining, by the processor, a plurality of δ's for each of the plurality of subjects, wherein each δ is a genetic change in the second sample genomic data compared to the first sample genomic data;
creating, by the processor, a matrix of the plurality of subjects and their features, wherein the features comprise the genetic changes or clusters of genetic changes in the plurality of δ's for each of the plurality of subjects;
biclustering, by the processor, the matrix of the plurality of subjects and their features, to provide clumps of subjects, each clump of subjects sharing a common feature, wherein the common feature is a shared genetic change or shared cluster of genetic changes; and
outputting, by the processor, the clumps of subjects, the common features, and the treatment.

2. The computer-implemented method of claim 1, further comprising permuting, by the processor, the matrix of subjects and their features and re-biclustering, by the processor, the permuted matrix to provide permuted clumps of subjects.

3. The computer-implemented method of claim 1, further comprising connecting, by the processor, the clumps of subjects by a feature edge for clumps that share features, a subject edge for clumps that share subjects, or a combination thereof

4. The computer-implemented method of claim 1, further comprising correlating the common feature and a phenotype of the clump of subjects.

5. The computer-implemented method of claim 1, wherein the genomic data is from the genome of the subjects, the first and second samples are biopsy samples, and the treatment is a cancer treatment; or wherein the genomic data is from the microbiome of the subjects, the first and second samples are gastrointestinal samples, and the treatment is antibiotic treatment, cancer treatment, and/or immunotherapy.

6. The computer-implemented method of claim 1, further comprising identifying, by the processor, a common mechanism of response to the treatment based on the common feature.

7. The computer-implemented method of claim 1, further comprising comparing, by the processor, genomic data for a new patient subjected to the treatment with the common feature for the clump of subjects, and if the new patient genomic data shares the common feature, determining that the new subject and the clump of subjects have a same mechanism of response to the treatment.

8. The computer-implemented method of claim 7, further comprising determining, by the computer, a further treatment for the new patient based upon the mechanism of response to the treatment.

9. The computer-implemented method of claim 8, further comprising administering the further treatment to the subject.

10. The computer-implemented method of claim 1, wherein determining, by the processor, the δ's, further comprises determining, by the processor, a noise threshold for the δ's based on an overall distribution of δ values.

11. The computer-implemented method of claim 7, wherein determining, by the processor, the noise threshold for the δ's comprises determining, by the processor, a p-value for the δ's, or determining, by the processor, a lower bound for the δ's.

12. The computer-implemented method of claim 1, comprising, prior to creating the matrix of the plurality of subjects and their features, binarizing, by the computer, the δ's.

13. The computer-implemented method of claim 1, wherein the genetic change comprises a presence of at least one gene; an absence of at least one gene; a sequence variation of at least one gene; or an expression level change of at least one gene.

14. A computer program product for generating a common feature resulting from a cancer treatment, the computer program product comprising a computer readable storage medium having program instructions embodied therewith, the program instructions executable by a processor to cause the processor to perform operations comprising:
inputting, to a processor, genomic data from a plurality of subjects, wherein the genomic data for each subject of the plurality of subjects comprises first sample genomic data from a first sample taken prior to a treatment, and second sample genomic data from a second sample taken after the treatment;
determining, by the processor, a plurality of δ's for each of the plurality of subjects, wherein each δ is a genetic change in the second sample genomic data compared to the first sample genomic data;
creating, by the processor, a matrix of the plurality of subjects and their features, wherein the features comprise the genetic changes or clusters of genetic changes in the plurality of δ's for each of the plurality of subjects;
biclustering, by the processor, the matrix of the plurality of subjects and their features, to provide clumps of subjects, each clump of subjects sharing a common feature, wherein the common feature is a shared genetic change or shared cluster of genetic changes; and
outputting, by the processor, the clumps of subjects, the common features, and the treatment.

15. The computer program product of claim 14, wherein the operations further comprise permuting, by the processor, the matrix of subjects and their features and re-biclustering, by the processor, the permuted matrix to provide permuted clumps of subjects.

16. The computer program product of claim 14, wherein the operations further comprise connecting, by the processor, the clumps of subjects by a feature edge for clumps that share features, a subject edge for clumps that share subjects, or a combination thereof.

17. The computer program product of claim 14, wherein the operations further comprise determining, by the processor, the δ's, further comprises determining, by the processor, a noise threshold for the δ's based on an overall distribution of δ values.

18. The computer program product of claim 17, wherein determining, by the processor, the noise threshold for the δ's comprises determining, by the processor, a p-value for the δ's, or determining, by the processor, a lower bound for the δ's.

19. The computer program product of claim 14, wherein the operations further comprise , prior to creating the matrix of the plurality of subjects and their features, binarizing, by the computer, the δ's.

20. A system for generating a common feature resulting from a cancer treatment comprising:
a processor; and
a computer readable storage medium storing comprising executable instructions that, when executed by the processor, cause the processor to perform operations comprising:
inputting, to a processor, genomic data from a plurality of subjects, wherein the genomic data for each subject of the plurality of subjects comprises first sample genomic data from a first sample taken prior to a treatment, and second sample genomic data from a second sample taken after the treatment;
determining, by the processor, a plurality of δ's for each of the plurality of subjects, wherein each δ is a genetic change in the second sample genomic data compared to the first sample genomic data;
creating, by the processor, a matrix of the plurality of subjects and their features, wherein the features comprise the genetic changes or clusters of genetic changes in the plurality of δ's for each of the plurality of subjects;
biclustering, by the processor, the matrix of the plurality of subjects and their features, to provide clumps of subjects, each clump of subjects sharing a common feature, wherein the common feature is a shared genetic change or shared cluster of genetic changes; and
outputting, by the processor, the clumps of subjects, the common features, and the treatment.
